(19) United States
(12) Patent Application Publication
(10) Pub. No.: US 2018/0093024 A1
(43) Pub. Date: Apr. 5, 2018

(54) BREAST PUMP MILK CAPTURE AND COLLECTION SYSTEM

(71) Applicant: Moxxly, Inc., San Francisco, CA (US)

(72) Inventors: Santhi Analytis, San Francisco, CA (US); Cara Delzer, San Francisco, CA (US); Gabriellle Guthrie, San Francisco, CA (US); Wisit Jirattigalachote, Palo Alto, CA (US); Jacob Kurzrock, San Francisco, CA (US); Sam Levey, Pittsburgh, PA (US); Claire O'Malley, San Francisco (US)

(73) Assignee: Moxxly, Inc., San Francisco, CA (US)

(21) Appl. No.: 15/286,207

(22) Filed: Oct. 5, 2016

(51) Int. Cl. A61M 1/06

(52) U.S. Cl. CPC A61M 1/062; A61M 1/066

(57) ABSTRACT

This disclosure generally relates to a breast pump valve system. The breast pump valve system includes a valve and a milk collection apparatus. The valve includes one or more retaining ribs which are disposed around the circumference of the valve. The one or more retaining ribs are interrupted only by a vent channel. The valve further includes a vent, connected to the vent channel. The vent channel allows air in the milk collection apparatus to escape through the vent in the valve. The milk collection apparatus includes a chamfered collar to receive the valve. The milk collection apparatus further includes a connector disposed on an external surface of the chamfered collar.

### BACKGROUND

### 1. Technical Field

This disclosure relates generally to a breast pump for nursing mothers. More specifically, the devices and systems disclosed herein relate to a valve disposed within a milk collection system for maintaining vacuum pressure while allowing milk to be collected in a milk collection apparatus.

### 2. Description of the Related Art

Nursing an infant can be an emotional experience for many nursing mothers. While nursing can be profoundly rewarding for a new mother, many mothers have difficulty nursing a new baby. Breast pumps have been developed to help women express milk by mechanical systems to feed to their babies. To that end, breast pumps have been developed to not only assist mothers who experience difficulty in nursing infants, but also to allow mothers who are away from their babies provide milk for their infants to drink at another time and help many mothers maintain and develop their milk supply.

Conventional breast pump systems are not well suited for today's user. These breast pumps have obtrusive parts that require a mother to undress to use the pump. Further, each individual part must be assembled before each use and cleaned after each use. This assemblage of parts frequently results in milk spills and unneeded frustration for a new mother. Since the likelihood of milk spills are increased when a significant number of parts must be assembled and disassembled, it is undesirable to have a large number of parts. Furthermore, many parts are small and easy to lose. If a mother forgets to bring one piece of the breast pump to work or on a trip with her, then the whole system will not work.

One conventional breast pump allows milk to drain through a hole covered by a flap. When the vacuum in the pump is engaged, the flap is drawn to the hole, covering and sealing the hole. Milk that is expressed during the vacuum cycle is allowed to pass through the hole when the vacuum pressure is released and the flap loses its seal to the hole. Milk is drained into a milk collection apparatus, which must be removed from the pump after pumping. Conventional milk collection apparatuses also provide no milk spillage solutions. For example, many mothers find it difficult to unthread a milk collection apparatus from the rest of the breast pump, transport the milk collection apparatus to a stable surface, and seal the milk collection apparatus without spilling at least a portion of the milk collected by the milk collection apparatus. Spilled milk represents wasted time and effort. More importantly, spilled milk is milk that a new mother cannot feed to her baby.

It is therefore one object of this disclosure to provide a milk collection apparatus that includes a valve that prevents the spillage of milk. A second object of this disclosure is to provide a valve that is large and visible, and completes the system in an integral way. It is a further object of this disclosure to provide a breast pump that minimizes the number of parts required to express and collect milk. Another object of this disclosure is to provide a valve in a milk collection apparatus that allows milk to drain into the milk collection apparatus while simultaneously sealing milk within the milk collection apparatus in a manner that prevents spillage.

### SUMMARY

Disclosed herein is a breast pump valve that includes one or more retaining ribs which are disposed around the circumference of the valve and are only interrupted by a vent channel disposed. The valve further includes a vent which is connected to the vent channel.

Further disclosed herein is a breast pump valve system. The breast pump valve system includes a valve and a milk collection apparatus. The valve includes one or more retaining ribs which are disposed around the circumference of the valve. The one or more retaining ribs are interrupted only by a vent channel disposed in the lower portion of the valve. The valve further includes a vent, connected to the vent channel, which is disposed in the upper portion of the valve. The vent channel allows air in the milk collection apparatus to escape through the vent in the valve. The milk collection apparatus includes a chamfered collar to receive the valve. The milk collection apparatus further includes a connector disposed on an external surface of the chamfered collar.

### BRIEF DESCRIPTION OF THE DRAWINGS

The accompanying drawings illustrate one or more embodiments of a breast pump implementing a milk collection valve system.

FIG. 1 illustrates a breast pump implementing a milk collection valve system.

FIG. 2 illustrates a milk collection apparatus used with the milk collection valve system.

FIG. 3 illustrates a valve used in conjunction with the milk collection apparatus in the milk collection valve system.

FIG. 4 illustrates a cross sectional side view of a valve implemented in the milk collection apparatus in the milk collection valve system.

FIG. 5 illustrates a second cross sectional side view of the valve implemented in the milk collection apparatus in the milk collection valve system shown in claim 4 rotated by 90°.

FIG. 6A illustrates another embodiment of a valve implemented in the milk collection apparatus in the milk collection valve system.

FIG. 6B illustrates the embodiment of the valve implemented in the milk collection apparatus in the milk collection valve system where the valve is reversed to act as a feeding nipple.

### DETAILED DESCRIPTION OF PREFERRED EMBODIMENTS

In the following description, for purposes of explanation and not limitation, specific techniques and embodiments are set forth, such as particular techniques and configurations, in order to provide a thorough understanding of the device disclosed herein. While the techniques and embodiments will primarily be described in context with the accompanying drawings, those skilled in the art will further appreciate that the techniques and embodiments may also be practiced in other similar devices.

Reference will now be made in detail to the exemplary embodiments, examples of which are illustrated in the accompanying drawings. Wherever possible, the same reference numbers are used throughout the drawings to refer to the same or like parts. It is further noted that elements disclosed with respect to particular embodiments are not restricted to only those embodiments in which they are described. For example, an element described in reference to one embodiment or figure, may be alternatively included in another embodiment or figure regardless of whether or not those elements are shown or described in another embodiment or figure. In other words, elements in the figures may be interchangeable between various embodiments disclosed herein, whether shown or not.

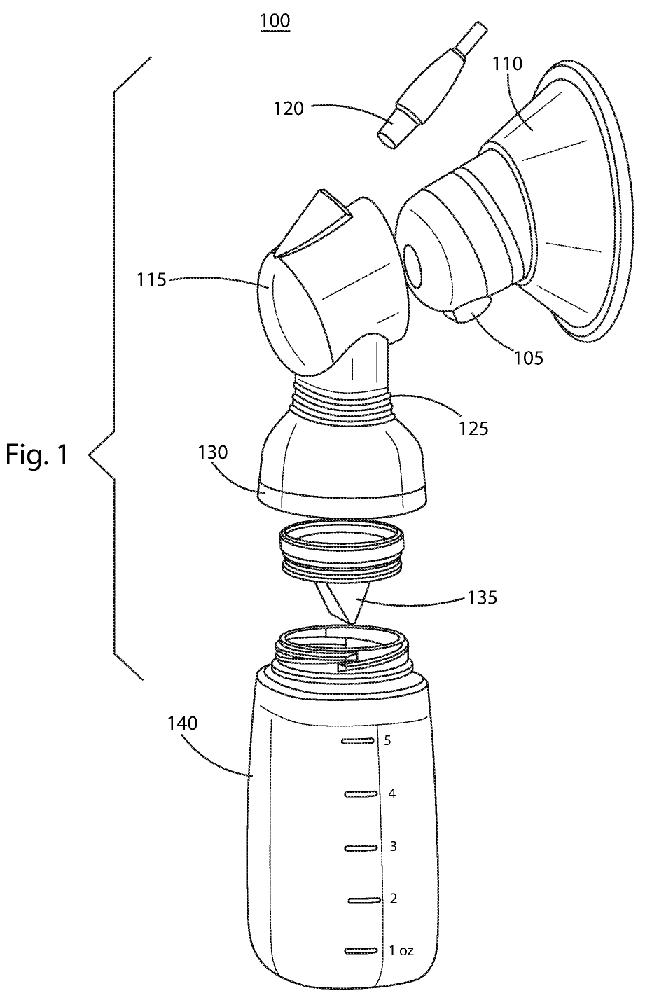

FIG. 1 illustrates a breast pump 100. Breast pump 100 includes a flange 105, which includes a support cone 110 that connects to manifold 115 using an airtight tapered friction fitting. Flange 105 is the breast-worn component of the system. Flange 105 may be constructed in various sizes, in order to accommodate users of different sizes. Inner diameters of flange 105, which contacts the user's areola during pumping, may range from 19 mm to 36 mm, with typical values at 21 mm, 24 mm, 27 mm, and 30 mm. The outer diameter of the support cone may be between 32 mm and 34 mm for all flange sizes, such that they mate with a single size manifold 115. In one embodiment, flange 105 may provide a user with tactile feedback with a step on support cone 110 to meet the manifold 115 in a manner that helps the user correctly align the flange 105 with manifold 115. Manifold 115 may house a number of elements, which are not shown in FIG. 1, including computer hardware, one or more processors, various types of memory (static, dynamic, flash, etc), receivers, transmitters, antennas, various sensors, a vacuum pump, and other elements that make breast pump 100 capable of interacting with, for example, a mobile device, such as a mobile phone. A vacuum pump, not shown, may also be a component that is external to breast pump 100 and may connect to breast pump 100 via tubing adapter 120 attached to manifold 115. Tubing adapter 120 may also provide tactile feedback to the user to ensure that pneumatic tubing (not shown) is properly connected to manifold 115. Tactile feedback may be provided by a series of mechanical stops installed in the various elements of breast pump 100 that provide the user a sense that the elements of breast pump 100 are correctly assembled. In order to enhance the comfort of breast pump 100, manifold 115 includes a flexible neck 125 which may remain flexible or may be flexed into a particular position and held there by flexible neck 125. The neck may allow for bending between 20 to 70 degrees from the vertical, as aligned with the user's torso. The flexible neck 125 may be made by co-molding 30 to 60 Shore A durometer thermoplastic elastomer (TPE) with rigid plastic which forms the manifold top 115 and connector portions 130.

Manifold 115 includes a manifold base 130 which includes a connector (not shown) that connects to a milk collection apparatus 140 which houses valve 135. In one embodiment, the connector in manifold base 130 may include female threads to receive male threads on milk collection apparatus 140. Alternatively, manifold base 130 may include a friction connector, which allows milk collection apparatus 140 to “snap” into manifold base 130. In a further embodiment, the connector in manifold base 130 may include a specific thread pattern to allow milk collection apparatus 140 to be fully secured by a quarter turn connection. More specifically, the connector in manifold base 130 may allow milk collection apparatus 140 to be fully secured to manifold base 130 by threading milk collection apparatus 140 into the connector in manifold base 130 and turning milk collection apparatus 140 90°. The user is visually and tactilely cued by the parts fitting together.

In practice, breast pump 100 is applied to a mother's breast by securing support cone 110 to a nipple area of the mother's breast. Vacuum pressure may then be applied by a vacuum pump through tubing 120 connected to manifold 115. As the vacuum pump cycles between an on state and an off state, milk is expressed from the mother's breast. The milk is allowed to drain through support cone 110 into manifold 115 and into valve 135. Valve 135 allows the milk to selectively drain in to milk collection apparatus 140. In one embodiment, valve 135 may be constructed using liquid injection silicone. The silicone may be between 30 and 70 Shore A durometer. As will be further discussed below, valve 135 includes an opening which allows milk to drain from valve 135 into milk collection apparatus 140. At the same time, however, valve 135 maintains the vacuum created within support cone 110, and manifold 115 by selectively opening and closing in response to vacuum pressure. For example, when vacuum pressure is being applied to a mother's breast, the vacuum pressure forces valve 135 to close and maintain the vacuum. When the vacuum pressure is off, valve 135 opens and allows milk to flow into milk collection apparatus 140. As the duty cycle of the vacuum pump rapidly alternates between application of vacuum pressure to the mother's breast and not applying vacuum pressure to the mother's breast (to simulate a suckling infant), valve 135 selectively responds to maintain vacuum pressure or allow milk to flow into milk collection apparatus 140.

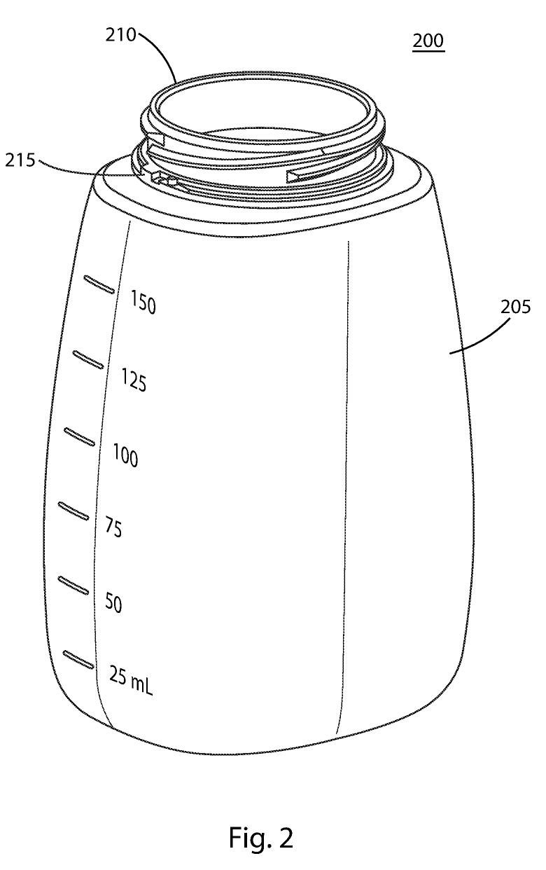

FIG. 2 illustrates a milk collection apparatus 200 used with a breast pump, such as breast pump 100 shown and described above with respect to FIG. 1. Milk collection apparatus 200 may be implemented as a bottle, such as bottle 205 and include fluidic measurement indicia for measuring the amount of milk within bottle 205. Bottle 205 preferably includes a flat base such that bottle 205 is stable on flat surfaces. Milk collection apparatus 200 includes an opening 210 which may be chamfered on an angle between 1 and 80 degrees. This chamfering on opening 210 allows a valve, such as valve 135 shown in FIG. 1, to be installed in opening 210 in a manner that maintains vacuum pressure applied within, for example, breast pump 100 shown in FIG. 1. The chamfer also provides improved sealing against a gasket in the lid during storage and transportation of milk. As discussed above, valve 135 is constructed using, for example, liquid injection molded silicone. The properties of liquid injection molded silicone allow valve 135 to be somewhat pliable while still being rigid enough to be supported by opening 210 of milk collection apparatus 200. The chamfering in opening 210 of milk collection apparatus 200 allows valve 135 to form an airtight seal between valve 135 and milk collection apparatus 200 thus preserving vacuum pressure created within breast pump 100. Opening 210 may transition from a chamfered edge into a straight or ribbed collar that provides a smooth resting surface for valve 135 and a corresponding external surface for connector 215. The collar of opening 210 will be discussed in further detail below.

Milk collection apparatus 200 may further include a connector 215 which is similar to the male connector on milk collection apparatus 140, shown in FIG. 1 and as discussed above. In FIG. 2, connector 215 is shown as a quarter turn thread engagement mechanism which allows milk collection apparatus 200 to be fully secured within, for example, manifold base 130 also shown in FIG. 1, by turning milk collection apparatus 200 90°.

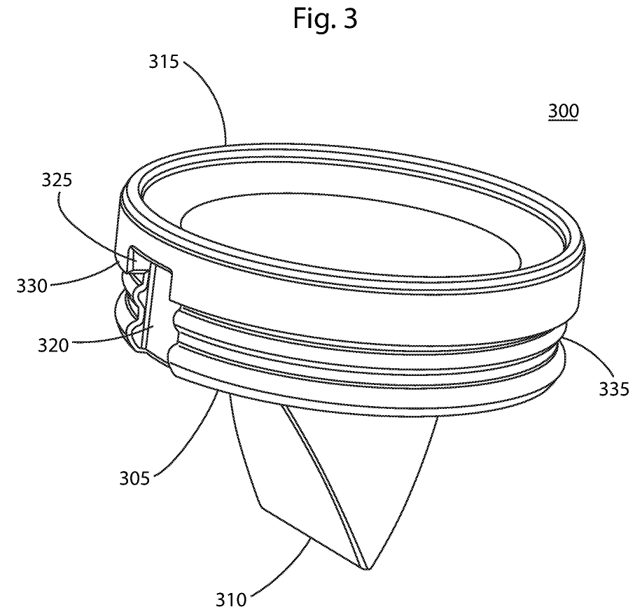

FIG. 3 illustrates a valve 300 used in conjunction with milk collection apparatus 200 shown in FIG. 2 and described above. Valve 300 is shown as being circular shaped and includes a funnel implemented as a valve spout 305 which funnels milk to valve opening 310. Valve 300 need not be strictly circular and may be implemented as an oval or any other shape. As discussed above, valve 300 may be implemented using liquid injection molded silicone and may taper along valve spout 305 to a relatively thin die cut opening to separate two or more leaflets of valve 300 into valve opening 310. The opening in the valve closes at 1 inHg or greater vacuum pressure but may function in vacuums of up to 25 inHg pressures. Typical vacuum ranges for causing the expression of milk from a breast are between 5-10 inHg. Thus, valve 300 may open at vacuum pressures less than 1 inHg and close at vacuum pressures greater than 1 inHg. More simply put valve 300 operates by vacuum pressure being selectively applied to valve 300 to cause valve 300 to open or close.

The length of the valve leaflets is between 5 mm and 30 mm in the preferred embodiment. The width of the valve leaflets is between 10 mm and 40 mm in the preferred embodiment. The opening silt at the leaflet tips is shorter than the width of the leaflet, by at least 1 mm to 3 mm. This small opening on a wide leaflet configuration reduces collapsing of the strutted sides of leaflets. Typically when the strutted sides collapse when opening and closing with the vacuum pressure changes, it causes a popping noise. This noise is minimized by the small opening size.

For pairs of leaflets, one leaflet is thicker than the other leaflet, for example 0.3 mm versus 0.5 mm, 0.2 mm versus 0.4 mm or some combination of ranges between 0.15 mm and 0.75 mm. This reduces high pitched vibrations that occur with the vacuum pressure changes during pumping. Typically, a breast pump vacuum oscillates between 0.5 Hz to 3 Hz, and the repetitive stops and starts of the suction causes the leaflets of a valve to vibrate near a resonance frequency. The quick opening and closing can cause honking noises. Having mismatched leaflet thicknesses changes the mass of the system, thus affecting the harmonics of the leaflet and can silence the honking or high pitched noises of the leaflets moving.

Valve 300 further includes a gasket 315 installed on a top edge or top surface of valve 300 and around the circumference of valve 300. Gasket 315 may be implemented as an integrated o-ring or a series of concentric o-rings, and serves to provide an airtight attachment between valve 300 and manifold base 130 shown in FIG. 1. When valve 300 is installed in, for example, milk collection apparatus 200 shown in FIG. 2 or milk collection apparatus 140 shown in FIG. 1, the connector, which is similar in description to connector 215 shown in FIG. 2, connects and tightens valve 300 between manifold base 130 and milk collection apparatus 200. This tightening action between the connector of milk collection apparatus 200, for example, and manifold base 130, for example, compresses gasket 315 and ensures an airtight seal between manifold 115 and milk collection apparatus 200.

Valve 300 further includes a vent channel 320. Vent channel 320 allows air from the ambient atmosphere to flow into milk collection apparatus 200, shown in FIG. 2, for example. However, while vent channel 320 allows air from the ambient atmosphere to flow into milk collection apparatus 200, vent channel 320 terminates before gasket 315 to ensure that ambient air does not flow into the vacuum sealed portion of breast pump 100, shown in FIG. 1. Vent channel 320 may include a vent 325, disposed above a top surface of milk collection apparatus 200 and above a valve stop 330. It should also be noted that many vents 325 may be implemented with many valve channels 320. Valve stop 330 allows a portion of valve 300 to be inserted into milk collection apparatus 200 while ensuring that the vent channel 320 provides air flow out of the milk collection apparatus 200 without adversely affecting the seal between valve 300 and milk collection apparatus 200 and the seal between valve 300 and manifold base 130, shown in FIG. 1. Vent 325 must be large enough for air to escape the bottle during pumping, yet in the preferred embodiment, vent 325 is small enough such that breast milk does not spill out in case the milk collection apparatus is knocked over or tilted during disassembly. The preferred width of vent 325 is between 1 mm and 6 mm, and the preferred depth of vent channel 320 is between 0.5 mm and 3 mm. Valve stop 330 is implemented as a ridge disposed around valve 300 that matches, approximately, the outside diameter of milk collection apparatus 200. As valve 300 is inserted into, for example, opening 210 of milk collection apparatus 200, valve stop 330 ensures that valve 300 will be inserted in such a manner as to ensure that vacuum pressure is maintained within manifold 115 of breast pump 100 in FIG. 1 while allowing air that is displaced in milk collection apparatus 200 by milk to vent out. One advantage of vent channel 320 is that should a bottle be knocked over or dropped, the milk contained within milk collection apparatus 200 will have a very small pathway from which to spill out of milk collection apparatus 200. Surface tension of falling milk seals the air vent hole. So long as valve 300 is installed on milk collection apparatus 200, the likelihood of milk spillage during and subsequent to disconnection of milk collection apparatus 200 from breast pump 100 is drastically reduced over conventional solutions. Further, any amount of milk that does spill through vent 325 will be negligible. The top of the air vent 325, may be wider than channel 320, in order to allow for a notch for easy removal, more aptly called an “affordance feature.” Similarly, other affordance features, such as separate notches in valve stop 330 may be used by the user to easily grasp, pinch and lift off the valve from the bottle top.

Valve 300 further includes one or more ribs 335 disposed under valve stop 330. Ribs 335 may be referred to simply as ribs or as “retaining ribs 335.” Two ribs 335 are shown in FIG. 3 but any number of ribs could be implemented on valve 300. Ribs 335 is a raised portion of liquid injection molded silicone, for example, that mates with the collar of opening 210 of milk collection apparatus 200 except at vent channel 320. Alternatively, ribs 335 may include a single rib and another rib, also called a lip, that extends over the opening 210 of milk collection apparatus 200 and attaches by elasticity to opening 210 of milk collection apparatus 200 to ensure that valve 300 stays in place when valve 300 is not connected to manifold base 130, shown in FIG. 1, for example. Ribs 335, therefore, secure valve 300 to milk collection apparatus 200 to provide a top or a lid to milk collection apparatus 200 both during pumping and during transport. Ribs 335 therefore allows milk to be securely held in place while also allowing vent channel 320 to channel air displaced by milk out of milk collection apparatus 200. In short, ribs 335 acts as a retainer to maintain valve 300 on/in milk collection apparatus 200.

Valve 300 consists of an upper portion and a lower portion which may also be referred to as an “above the bottle portion” and a “below the bottle portion.” Specifically, those elements of the valve that are disposed above valve stop 330 (and including valve stop 330) when the bottle is installed comprise the upper portion of valve 300. Similarly, those elements of the valve that are disposed below valve stop 330 comprise the lower portion of valve 300. Vent channel 320, for example, may be disposed in the lower portion of valve 300 and connect to vent 325 in the upper portion of valve 300. The upper portion of the valve including the valve stop is between 3 mm and 20 mm in the preferred embodiment.

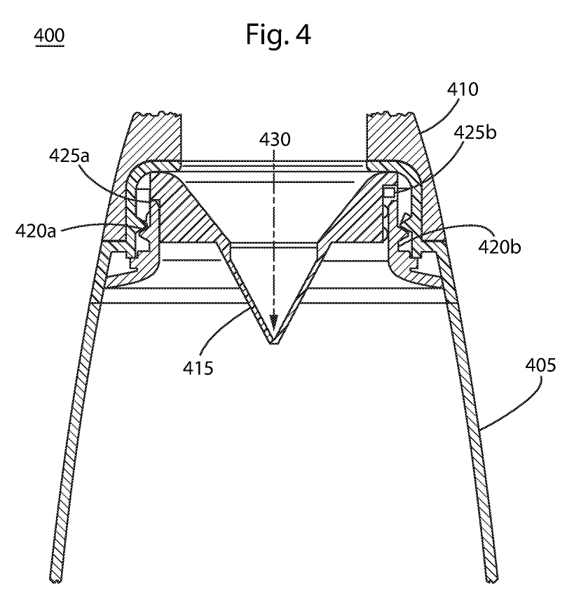

FIG. 4 illustrates a cross sectional side view of a valve 415 implemented in the milk collection apparatus 405 in milk collection valve system 400. Milk collection valve system 400 includes a milk collection apparatus 405, implemented as a bottle in FIG. 4. Milk collection apparatus 405 is similar in description to milk collection apparatus 200 shown in FIG. 2 and milk collection apparatus 140 shown in FIG. 1. Valve 415 is similar to valve 300 shown and discussed above with respect to FIG. 3. Valve 415 is installed into milk collection apparatus 405 by inserting valve 415 into milk collection apparatus 405. Valve 415 further includes valve stop 425a, which is similar to valve stop 330 discussed above with respect to FIG. 3. Thus valve 415 is installed by inserting valve 415 such that valve stop 425a sits on the collar of milk collection apparatus 405. One or more ribs are deformed in FIG. 4, such as the one or more ribs shown in FIG. 3 as one or more ribs 335, because they are mated with the collar of milk collection apparatus 405, ensuring that valve 415 remains in place to prevent milk spillage.

Once valve 415 is installed on milk collection apparatus 405, milk collection apparatus 405 is installed by threaded quarter lock connection to manifold 410, which is similar in implementation and description to manifold 115 and manifold base 130, shown in FIG. 1 and described above. Once milk collection apparatus 405 is securely connected to manifold 410 by connectors 420a and 420b, valve 415 is correctly positioned with vent 425b exposed to ambient air. Connectors 420a and 420b may be implemented with a quarter turn engagement thread mechanism. Accordingly, milk may follow path 430 to valve 415 and flow into milk collection apparatus 405 as valve 415 opens and closes as described above.

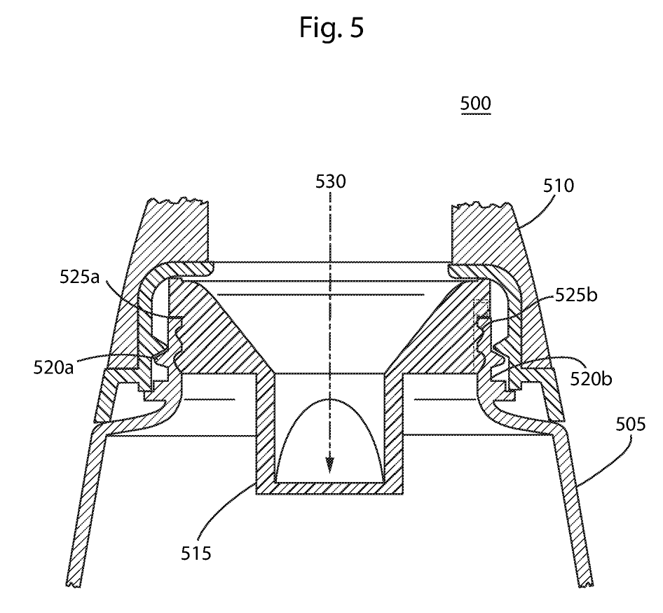

FIG. 5 illustrates a second cross sectional side view of valve 515 implemented in the milk collection apparatus 505 in the milk collection valve system 400 shown in claim 4 rotated by 90° to demonstrate milk collection valve system 500. Milk collection valve system 500 includes a milk collection apparatus 505, implemented as a bottle in FIG. 5. Milk collection apparatus 505 is similar in description to milk collection apparatus 200 shown in FIG. 2 and milk collection apparatus 140 shown in FIG. 1. Valve 515 is similar to valve 300 shown and discussed above with respect to FIG. 3. Valve 515 is installed into milk collection apparatus 505 by inserting valve 515 into milk collection apparatus 505. Valve 515 further includes valve stop 525a, which is similar to valve stop 330 discussed above with respect to FIG. 3. Thus valve 515 is installed by inserting valve 515 such that valve stop 525a sits on the collar of milk collection apparatus 505. One or more ribs are shown as ribs 525b that correspond in both implementation and description to ribs 335 shown in FIG. 3. In FIG. 5, ribs 525b are shown installed in two corresponding grooves formed in the collar of milk collection apparatus 505. In this manner, friction between ribs 525b and grooves in the collar of milk collection apparatus 505 is enhanced providing a tighter and more positive connection between ribs 525b and milk collection apparatus 505. Milk collection valve system 500 may include one or more ribs 525b and one or more corresponding grooves in milk collection apparatus 505 as necessary. Also, as discussed above, ribs 525b are disposed around the circumference of milk collection valve system 500 except where the vent (not shown in FIG. 5) is located along the circumference of milk collection valve system 500. In other words, ribs 525b are only broken around the circumference of milk collection valve system 500 by the vent, such as vent 425b, shown in FIG. 4.

Once valve 515 is installed on milk collection apparatus 505, milk collection apparatus 505 is installed by threaded quarter lock connection to manifold 510, which is similar in implementation and description to manifold 115 and manifold base 130, shown in FIG. 1 and described above. Once milk collection apparatus 505 is securely connected to manifold 510 by connectors 520a and 520b, valve 515 is correctly positioned with the vent (not shown in FIG. 5) exposed to ambient air. Connectors 520a and 520b may be implemented with a quarter turn engagement thread mechanism. Accordingly, milk may follow path 530 to valve 515 and flow into milk collection apparatus 505 as valve 515 opens and closes as described above.

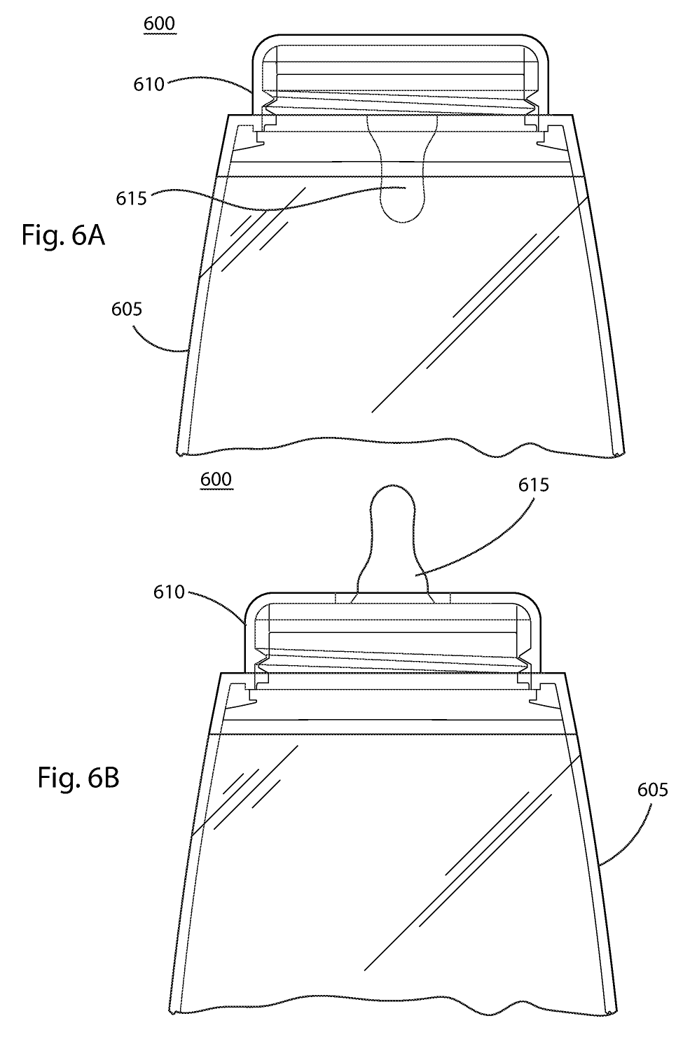

FIG. 6A illustrates another embodiment of a valve 615 implemented in milk collection apparatus 605 in a milk collection valve system 600. In FIG. 6a, valve 615 is implemented as a standard feeding nipple and is attached via connector 610. Rather than being a die cut opening to separate two or more leaflets, valve 615 may have a small circular opening or one or more slits or cross slits. In this configuration, valve 615 operates in exactly the same manner as other valves described above. Alternatively, the nipple-shaped valve may have feature as described in FIGS. 3 through 5 to attach to the top of the milk collection apparatus, and connector 610 may be incorporated in the capture portion of the breast pump system.

One benefit of implementing a valve in the shape of a standard feeding nipple is that the valve may be reversible, as shown in FIG. 6B. More clearly, the standard feeding nipple may be inverted or reversed from its position as valve 615. In FIG. 6B, milk collection apparatus 605 in milk collection valve system 600 is connected by a connector 610 to valve 615. However, in this configuration, valve 615 may be used as a feeding nipple to feed an infant. In other words, valve 615 may be reversed such that an infant may suckle on the valve to draw milk out of milk collection apparatus 605 through valve 615. If the standard feeding nipple is reversed, another retaining ring may be provided with a connector similar to 610 to secure the standard feeding nipple to milk collection apparatus 605.

In another embodiment, the nipple valve may have a threaded portion that sits unused above the collar of the milk collection apparatus during pumping, however is turned around and threaded into the bottle for use during feeding.

Other accessories may be provided. For example, a lid, not shown, may be provided to secure a valve to the milk collection apparatus. The lid may further include a lip that mates with the chamfer on the opening of the milk collection apparatus to securely hold the valve in place during transport, thereby preventing milk from spilling.

Alternatively, a lid may be used without a valve in place. In this embodiment, the lid has an integrated gasket to mate with the chamfered opening of the milk collection apparatus. This mating seals the liquid contents in the milk collection apparatus and prevents spills during storage and transportation.

The foregoing description has been presented for purposes of illustration. It is not exhaustive and does not limit the invention to the precise forms or embodiments disclosed. Modifications and adaptations will be apparent to those skilled in the art from consideration of the specification and practice of the disclosed embodiments. For example, components described herein may be removed and other components added without departing from the scope or spirit of the embodiments disclosed herein or the appended claims.

Other embodiments will be apparent to those skilled in the art from consideration of the specification and practice of the disclosure disclosed herein. It is intended that the specification and examples be considered as exemplary only, with a true scope and spirit of the invention being indicated by the following claims.

What is claimed is:

1. A breast pump valve, comprising:
one or more retaining ribs which are disposed around the circumference of the valve wherein the one or more ribs are interrupted only by a vent channel; and
a vent, connected to the vent channel.

2. The breast pump valve of claim 1, further comprising:
a valve stop.

3. The breast pump valve of claim 2, wherein the valve stop is configured to mate with a collar of a milk collection apparatus.

4. The breast pump valve of claim 1, wherein the one or more ribs are implemented as two ribs.

5. The breast pump valve of claim 1, wherein the valve includes a funnel.

6. The breast pump valve of claim 1, further comprising two or more valve leaflets.

7. The breast pump valve of claim 6, wherein the two or more valve leaflets are die cut.

8. The breast pump valve of claim 6, wherein the leaflets open and close in response to vacuum pressure.

9. The breast pump valve of claim 8, wherein the vacuum pressure is between 1 inHg and 25 inHg.

10. The breast pump valve of claim 1, wherein the valve is formed using liquid injection molded silicone.

11. The breast pump valve of claim 1, further comprising a gasket implemented as one or more O-rings.

12. A breast pump valve system, comprising:
a valve including:
one or more retaining ribs which are disposed around the circumference of the valve wherein the one or more ribs are interrupted only by a vent channel disposed in the lower portion of the valve; and
a vent, connected to the vent channel, and disposed in the upper portion of the valve;
a milk collection apparatus including:
a collar to receive the valve; and
a connector disposed on an external surface of the collar
wherein the vent channel allows air in the milk collection apparatus to escape through the vent in the valve.

13. The breast pump valve system of claim 12, wherein the valve is inserted into the collar of the milk collection apparatus.

14. The breast pump valve system of claim 12, wherein the valve further includes a valve stop that mates with the collar of the milk collection apparatus.

15. The breast pump valve system of claim 12, wherein the connector uses a quarter turn thread engagement mechanism.

16. The breast pump valve system of claim 12, wherein the one or more retaining ribs are inserted in the collar of the milk collection apparatus.

17. The breast pump valve system of claim 12, wherein the valve is a standard feeding nipple.

18. The breast pump valve system of claim 12, wherein the valve is reversible.

19. The breast pump valve system of claim 12, wherein the valve includes one or more affordance feature.

20. The breast pump valve system of claim 11, further comprising a second valve channel and a second vent.
